(19) United States
(12) Patent Application Publication
(10) Pub. No.: US 2022/0313707 A1
(43) Pub. Date: Oct. 6, 2022

(54) USE OF AR INHIBITOR AND/OR HIF-1 A A INHIBITOR IN PREPARATION OF MEDICAMENT

(71) Applicant: Suzhou Molecular Intersection Biomedical Co., Ltd., Suzhou (CN)

(72) Inventors: Yifeng Zhou, Jiangsu (CN); Qiang Guo, Jiangsu (CN); Zheng Zhang, Jiangsu (CN); Binbin Guo, Jiangsu (CN); Siqi Wu, Jiangsu (CN)

(73) Assignee: Suzhou Molecular Intersection Biomedical Co., Ltd., Suzhou (CN)

(21) Appl. No.: 17/750,888

(22) Filed: May 23, 2022

Related U.S. Application Data
(63) Continuation of application No. PCT/CN2022/078698, filed on Mar. 2, 2022.

(30) Foreign Application Priority Data
Mar. 8, 2021 (CN) 202110248587.8

(51) Int. Cl. A61K 31/58; A61K 31/18; A61K 31/245; A61K 31/198; A61K 31/4166; A61K 31/4439; A61K 31/4155; A61K 31/5025; A61P 31/16; A61P 37/06

(52) U.S. Cl. CPC A61K 31/58; A61K 31/18; A61K 31/245; A61K 31/198; A61K 31/4166; A61K 31/4439; A61K 31/4155; A61K 31/5025; A61P 31/16; A61P 37/06

(57) ABSTRACT

The present disclosure discloses use of a combination of an HIP-1α inhibitor and/or an AR inhibitor in inhibiting an .inflammatory cytokine storm,. treating or preventing viral pneumonia. The present disclosure finds that inhibitors HIF-1 a and AR prevent the transcription of inflammatory cytokine storm-related genes (IL6, MMP2, MMP13, ADAMTS4, ELN, VCAN. COL3A1 and VEGFA) in fibroblasts under low-oxygen environment by effectively inhibiting the interaction between HIF-1α and AR to reduce the expression quantity of the related genes, thereby influencing the activation of lung fibroblasts. Inhibition of activation of lung fibroblasts enables patients with acute respiratory distress syndrome (ARDS) due to severe respiratory tract infection to maintain the lung functions and improve clinical efficacy.

### CROSS-REFERENCE TO RELATED APPLICATIONS

This application is a continuation-in-part of International Application No. PCT/CN2022/078698, filed on Mar. 2, 2022, which claims priority to Chinese Patent Application No. 202110248587.8, filed on Mar. 8, 2021, both of which are hereby incorporated by reference in their entireties.

### TECHNICAL FIELD

The present disclosure relates to the technical field of viral pneumonia therapy, and particularly the use of an AR inhibitor and/or an HIF-1α inhibitor in the preparation of a. medicament for inhibiting an inflammatory cytokine storm, in prevention or treatment of viral pneumonia.

### BACKGROUND

Severe respiratory tract infection can lead to acute respiratory distress syndrome (ARDS). At present, no effective medicament treatment has been demonstrated to improve prognosis of ARDS patients. Although the inflammatory response of a host limits the propagation of pathogens and ultimately clears away pathogens, immunopathogenesis is one of the major reasons that cause tissue injury and ARDS. Moreover, lung fibroblasts play an important role in viral pneumonia. Respiratory virus infection cause different activated. states of fibrocytes, including extracellular matrix (ECM) synthesis, damage response and interferon response state. During the severe influenza. virus infection, the overactivity of the lung fibroblasts in damage response induces fatal immunopathogenesis. The lung fibroblasts in damage response will change the lung microenvironment by producing ECM remodeling enzymes and inflammatory factors to promote the infiltration of immune cells; thereby, damaging pulmonary functions. A therapeutic agent targeting damage-response lung fibroblasts can provide a promising method to maintain pulmonary functions and improve clinical efficacy after severe respiratory tract infection.

Viral pneumonia is a pulmonary inflammation caused by upper respiratory tract viral infection and its further expansion downwards. Based on the severity of the symptoms, viral pneumonias are categorized into mild, moderate, severe and critical types. Mild eases have slight clinical symptoms and imaging manifestations of pneumonia cannot be detected; moderate cases are accompanied with fever, respiratory symptoms and imaging manifestations of pneumonia can be detected; severe cases suffer from hyoxemia and have static oxygen saturation less than 93% or PaO₂/FiO₂ less than 300 mmHg; critical cases require mechanical ventilation or ICU intensive care, etc. The critical point of judging the severity of viral pneumonia. lies in hyoxemia. Hyoxemia causes an anoxic environment to occur in the lungs; meanwhile, severe patients will rapidly progress ARDS, septic shock, irreformable metabolic acidosis, bleeding/coagulation disorders, multiple organ failure and the like.

The infection of viral pneumovirus will lead to ARDS to cause the inflammatory response of lung tissues; the multiple stimulatory substances generated by the inflammatory response make normal lung fibroblasts activated. Activated fibroblasts can produce a large number of inflammatory cytokines, e.g., interleukin 6, and matrix proteinases such as MMP2. Meanwhile, the activation of lung fibroblasts will further aggravate the clinical symptoms of viral pneumonia. It has been reported in the prior art that to explore and determine the upstream stimulation to activate lung fibroblasts of a patient with respiratory tract infection, human seasonal H3N2 virus or avian H5N6 and H7N9 viruses are used to in vitro infect normal human bronchial epithelial cells (NHBES), thus judging whether the virus is enough to drive the inflammatory transcription program in human lung fibroblasts during co-culture. The result indicates that the infection of NHBEs induces the expression of genes rich in inflammatory state in fibroblasts. These genes include interleukin 6 (IL6), matrix proteinases, e.g., matrix metalloproteinase 2 (MMP2), matrix metalloproteinase 13 (MMP13) and ADAMTS4, extracellular matrix protease elastin (ELN), versican (VCAN), type-III collagen gene α1 (COL3A1), vascular endothelial growth factors (VEGFA) and the like. It has been found by researches that the AR inhibitor Proxalutamide can target the AR-ACE2/TMPRSS2 signal axis, decrease or block COVID-19 from entering into host cells by inhibiting the expression of an angiotensin converting enzyme 2 (ACE2) and a transmembrane serine protease 2 (TMPRSS2). However, ACE2 and TMPRSS2 mainly influence the infection of COVID-19. Meanwhile, the failure of Reindesivir in clinical trials (NCT04257656) of COVID-19 severe cases also indicates that single antiviral infection cannot improve the turnover of severe patients. The most important reason to cause the viral pneumonia patients to become severe cases is hyoxemia. The low oxygen environment in lung tissues of severe patients leads to the high expression of HIF-1α in lung fibroblasts.

Therefore, it is very necessary for the treatment of viral pneumonia to seek a pharmacological method for regulating gene expression level and controlling fibroblast activation in an inflammatory cytokine storm, thus coming into play in the treatment of viral pneumonia.

### SUMMARY

To solve the above technical problems, an effective medicament directed to the treatment of viral pneumonia is screened. The inventor of the present disclosure finds in studies that HIF-1α in lung fibroblasts of viral pneumonia patients has a higher expression level. Moreover, the complex formed by interaction of HIF-1α and AR may be bound on promoters of related genes; thereby, influencing the expression of the related genes (IL6, MMP2, MMP13, ADAMTS4, ELN, VCAN, COL3A1 and VEGFA) in the transcriptional level.

In a first aspect, the present disclosure provides use of an AR inhibitor and/or an HIF-1α inhibitor in the preparation of a medicament for inhibiting an inflammatory cytokine storm. The AR inhibitor andlor the HIF-1α inhibitor inhibit the following in fibroblasts: gene expression activity of AR, gene expression activity of HIF-1α, as well as formation of an AR and HIF-1α complex.

In some examples, formation of an AR and HIF-1α complex in fibroblasts is inhibited to influence the expression of an inflammation-related gene, and the inflammation-related gene is selected from IL6, MMP2, MMP13, ADAMTS4, ELN, VCAN, COL3A1 and VEGFA.

In some examples, the inflammatory cytokine storm is caused by viral pneumonia.

In some examples, the inflammatory cytokine storm is moderate or severe.

In some examples, the HIF-1α inhibitor is selected from one or more of KC7F2, LW 6 and PX-478 2HCl.

In some examples, the AR inhibitor is selected from one or more of Enzalutamide, Apalutamide, Darolutamide, Proxalutamide, Galeterone, AZD3514 and SHR-3680.

In a second aspect, the present disclosure further provides use of an AR inhibitor andlor an HIF-1α inhibitor in the preparation of a medicament for preventing or treating viral pneumonia.

In some examples, the viral pneumonia is RSV (respiratory syncytial virus) pneumonia, influenza A virus pneumonia or COVID-19.

In some examples, the inflammatory cytokine storm is moderate or severe.

In some examples, the HIF-1α inhibitor is selected from one or more of KC7F2, LW 6 and PX-478 2HCl.

In some examples, the AR inhibitor is selected from one or more of Enzalutamide, Apalutamide, Darolutamide, Proxalutamide, Galeterone, AZD3514 and SHR-3680.

In a third aspect, the present disclosure further provides a method for screening an active compound inhibiting AR activation caused by HIF-1α, including the following steps:

Step S1—contacting HIF-1α with AR in the presence of a detected compound;

Step S2—detecting whether an interaction between HIF-1α and AR occurs;

Step S3—selecting a compound inhibiting the interaction in the step S2.

In a fourth aspect, the present disclosure further provides a method for treating or preventing viral pneumonia; the method includes administering a subject in need an effective amount of an AR inhibitor and/or an HIF-1α inhibitor.

In some examples, the viral pneumonia is respiratory syncytial virus pneumonia, influenza A virus pneumonia or COVID-19.

In some examples, the viral pneumonia is moderate or severe.

In some examples, the HIF-1α inhibitor is selected from one or more of KC7F2, LW 6 and PX-478 2HCl.

In some examples, the AR inhibitor is selected from one or more of Enzalutamide, Apalutamide, Darolutamide, Proxalutamide, Galeterone, AZD3514 and SHR-3680.

In some examples, a second therapeutic agent may be administered to a subject in need.

In some examples, the second therapeutic agent includes an antibody medicament, an antibiotic and the like.

In a fifth aspect, the present disclosure thither provides a medicator; the medicator includes a component for holding the AR inhibitor and/or the HIF-1α inhibitor or administering the AR inhibitor and/or the HIF-1α inhibitor to a subject, for example, a syringe, an infusion set or an implantable medicator.

In some examples, the medicator includes: an infusion module configured to administer a subject an AR inhibitor and/or HIF-1α inhibitor; an AR inhibitor and/or an HIF-1α inhibitor; and an efficacy monitoring module.

The present disclosure provides the following technical advantages over the prior art:

1) The present disclosure finds that the AR inhibitor and/or the HIF-1α inhibitor can prevent the transcription of related genes (IL6, MMP2, MMP13, ADAMTS4, ELN, VCAN, COL3A1 and VEGEA) in fibrdblasts under low-oxygen environment by effectively inhibiting the interaction between HIF-1α and AR to change the expression quantity of the related genes. The reduction of related gene expressions will influence the activation of lung fibroblasts such that patients with acute respiratory distress syndrome (ARDS) due to severe respiratory tract infection can maintain pulmonary functions and. improve clinical efficacy.

2) The medicament prepared by the HIF-1α inhibitor and AR inhibitor and used for inhibiting an inflammatory cytokine storm and preventing or treating viral pneumonia provided by the present disclosure have the following advantages: low costs, insignificant toxicity and side effects, and significant efficacy, thus providing a new strategy for treatment of viral pneumonia.

### BRIEF DESCRIPTION OF DRAWINGS

The summary of the present disclosure and the following detailed description of the present disclosure will be understood better with reference to the accompanying drawings. It should be understood that the present disclosure is not limited to the embodiments as shown in the accompanying drawings.

FIG. 1 shows expression quantity of HIF-1α in lung fibroblasts of a COVID-19 (Corona Virus Disease 2019) patient;

FIG. 2 is an enlarged diagram showing the expression quantity of HIF-1α in lung fibroblasts of the COVID-19 patient;

FIG. 3A shows expression levels of genes ACE2 and TMPRSS2 in lung fibroblasts of a COVID-19 patient and a subject not infected with COVID-19.

FIG. 3B shows expression levels of genes ACE2 and TMPRSS2 in lung fibroblasts in case of adding androgen for culture under anoxic and normoxia conditions.

FIG. 4 shows interaction between HIF-1α and AR forms a complex in human lung fibroblasts.

FIGS. 5A-5H show expression levels of related genes after being treated by different groups of inhibitors in Example 5.

FIGS. 6A-6H show expression levels of related genes after being treated by, different groups of inhibitors in Example 6.

FIGS. 7A-7B show statistics on days of mice survival and survival rate in Example 7.

### DETAILED DESCRIPTION OF EMBODIMENTS

### GENERAL OVERVIEW

The present disclosure discloses use of an HIF-1α inhibitor, an AR inhibitor or a combination thereof in inhibiting an inflammatory cytokine storm, treating or preventing viral pneumonia, especially in moderate and severe pneumonia. The inventor finds that inhibitors of HIF-1α and AR may prevent the transcription of inflammatory cytokine storm-related. genes (IL6, MMP2, MMP13, ADAMTS4, ELN, VCAN, COL3A1 and VEGFA) in fibroblasts under low-oxygen environment by effectively inhibiting the interaction between HIF-1α and AR to reduce the expression quantity of the related genes, thereby influencing the activation of lung fibroblasts, inhibition of activation of lung fibroblasts enables patients with acute respiratory distress syndrome (ARDS) due to severe respiratory tract infection to maintain pulmonary functions and improve clinical efficacy.

### DEFINITION OF TERMS

As used herein, the term “AR”, an androgen receptor, belongs to a steroid receptor in a nuclear receptor superfamily. AR generally consists of four domains: an N-terminal transactivation domain (NTD), a DNA binding domain (DBD), a hinge domain and a ligand binding domain (LBD). Human AR gene is located on X chromosome (Xq11-12), contains 8 exons and 7 introns with an overall length of 90 kb around. Androgen bind to AR in cytosol to work. After binding to androgen, the exertion and structure of AR will change and its physiolouicall functions—nuclear transport, transcription and phosphorylation—also update.

The term “androgen receptor (AR) inhibitor”refers to an active pharmaceutical ingredient capable of preventing or inhibiting the biological effect of androgen on in vivo normal reactive tissues.

The term “AR inhibitor”or “AR antagonist”can be used interchangeably herein, and refers to a compound or a combination to inhibit or reduce at least one activity of AR polypeptide. Exemplary AR activity includes but not limited to coactivator binding, DNA binding, ligand binding or nuclear translocation.

Exemplary “AR receptor antagonist”, “AR inhibitor”or “AR antagonist”includes but not limited to Enzalutamide, Apalutamide, Darolutamide, Proxalutarnide, Galeterone, AZD3514, SHR-3680, Flutamide, nilutamide, and bicalutamide.

Information on the structural, physical and chemical properties and pharmaceutical activity of Enzalutamide can be referring to No. CAS 9115087-33-1; information on the structural, physical and chemical properties and pharmaceutical activity of Apalutamide can be referring to No. CAS 1332391-92-0; information on the structural, physical and chemical properties and pharmaceutical activity of Darolutamide can be referring to No. CAS. 1297538-32-9; information on the structural, physical and chemical properties and pharmaceutical activity of Proxalutamide can be referring to No. CAS 1398046-21-3; information on the structural, physical and chemical properties and. pharmaceutical activity of Galeterone can be referring to No. CAS 851983-85-2; information on the structural, physical and chemical properties and pharmaceutical activity of Flutamide can be referring to No. CAS 13311-84-7; information on the structural, physical. and chemical properties and pharmaceutical activity of nilutamide can be referring to No. CAS 63612-50-0; information on the structural, physical and chemical properties and pharmaceutical activity of bicalutamide can be referring to No. CAS 90357-06-5; information on the structural, physical and chemical properties and pharmaceutical activity of AZD3514 can be referring to No. CAS 1240299-33-5; intbrmation on the structural, physical and chemical properties and pharmaceutical activity of SHR-3680 can be referring to No. CAS 1572045-62-5.

The term “HIF-1α”, hypoxia inducible factor-1 (HIF-1α) is a transcription factor extensively existing in mammal and human body under anoxic conditions, and is a key factor response to oxygen deprivation stress. HIF-1α is a subunit of hypoxia inducible factor-1 (HIF-1), regulated and controlled by oxygen deprivation and regulates the activity of HIF-1. HIF signal cascade reaction is under the influence of anoxic conditions in cells. HIF-1α transfers into cell nucleus to bind HIF-1β to form activated HIF-1 under anoxic conditions, thus regulating the transcription of multiple genes by binding to anoxic response elements on target genes. HIF-1α can constitute different signaling pathways together with multiple forward and reverse proteins to mediate low oxygen signals, regulate and control cells to produce a series of compensation responses on oxygen deprivation, which plays an important role in growth and development, physiological and pathological processes of body. HIF-1α is a focus in the study of biomedicine. HIF-1α consists of 826 amino acids; human HIF-1α gene is located in q21-24 regions of No. 14 chromosome. HIF-1α belongs to a basic-helix-loop-helix (bHLH)/PER-ARNT-SIM (PAS) protein fiimily. The N-terminal of HIF-1α contains a basic MIR configuration, which is an essential structure to bind DNA; the reverse proline-serine-threonine (Pro/Ser/Thr) is a specific structure to form a heterodimer and bind to target genes. The C-terminal of HIF-1α contains 3 domains; one is transactivation domain-terminal (TAD-C), which has the effect of regulating transcription. Another one is transactivation domain-N terminal (TAD-N), which is capable of activating transcription. The other one is oxygen-dependent degradation domain (ODDD), which is capable of degrading the HIF-1α protein by ubiquitination pathway. The C-terminal further has a nuclear localization signal (NLS) capable of assisting HIF-1α protein and nucleoporin to be bound into nucleus. The activation domain of the N-terminal interact with HIF-1β to form a heterodimer HIF-1, and then interact with cis-acting elements of hypoxia response elements (HRE) for transcription. The expression of HIF-1α cannot be detected basically under normal oxygen saturation; when oxygen concentration is lower than 5%, HIF-1α exists in cells stably. HIF-1α is rapidly degraded by ODDD-mediated ubiquitin proteasome pathway under normal oxygen saturation; however, under anoxic conditions, the degradation of HIF-1α is inhibited due to the reduced ubiquitination and hydroxylation levels.

As used herein, the term “HIF-1α inhibitor”refers to a compound or a composition capable of inhibiting AR activity. The HIF-1α inhibitor includes but not limited to one or more of KC7F2, LW 6, PX-478 2HCl, Oltipraz and Echinomycin. Information on the structural, physical and chemical properties and pharmaceutical activity of KC7F2 can be referring to No. CAS. 927822-86-4; information on the structural, physical and chemical properties and pharmaceutical activity of LW 6 can be referring to No. CAS 934593-90-5; information on the structural, physical and chemical properties and pharmaceutical activity of PX-478 2HCl can be referring to No. CAS 685898-44-6. Information on the structural, physical and chemical properties and pharmaceutical activity of Oltipraz can be referring to No. CAS 64224-21-1; information on the structural, physical and chemical properties and pharmaceutical activity of Echinomycin can be referring to No. CAS 512-64-1.

As used herein, the term “AR and HIF-1α complex”refers to a complex fOrmed by the interaction of HIF-1α with the N-terminal domain of androgen receptor (AR) through its own C-terminal activation domain

As used herein, the term “inflammatory cytokine storm”is also called cytokine storm, which refers to a phenomenon of rapidly producing a variety of cytokines in body fluid after a body is infected with microorganisms (the cytokines being such as tumor necrosis factor-alpha, interleukin-1, interleukin-6, interleukin-12, interferon alpha, interferon beta and interferon gamma), and is an important reason to cause ARDS and multiple organ failure. The cytokine storm may include inappropriate immunoreaction produced by a positive feedback loop between cytokines and immune cells. Symptoms of the cytokine storm may include high fever, red and swelling, fatigue and nausea. Although daily work of immune system is to clear away infection, however, if the immune system is activated to a degree of limit or out of control, it will do damage to the host.

As used herein, the term “fibroblast”refers to cells with vigorous functional activities; cell and cell nucleus are larger and clear in outline; nucleolus is large and Obvious; cytoplasm has weak basophilia, and fibrOblasts have obvious protein synthesis and secretory activities. Fibroblasts in mature period or resting state, which are called fibrocytes, decrease in volume and are of a long shuttle shape, and have underdeveloped rough endoplasmic reticulum and Golgi complex. Stimulated by trauma and other factors, a portion of fibrocytes can be transformed into immature fibrobla.sts again and functional activities also get recovered, thus participating in the repairing of tissue injury.

The term “viral pneumonia”refers to pneumonia caused by virus infection. Common viruses causing viral pneumonia include, for example, influenza virus, parainfluenza virus, adenovirus, coronavirus, rhinovirus, respiratory syncytial virus and interstitial pneumonia virus. Influenza A virus and influenza B virus mainly cause human flu. Influenza A virus usually has antigenic variations, thus being further categorized into subtypes such as H1N1, H3N2, H5N1 and H7N9. Moreover, human diseases mainly caused by coronavirus (belonging to Caronaviridae of Nidovirale) are respiratory tract infections. The respiratory tract infection is the major reason to the morbidity and mortality of viral pneumonia COVID-19, also called Corona Virus Disease 2019, is a disease caused by severe acute respiratory syndrome coronavirus 2 (SARS-CoV-2); SARS-CoV-2 is an enveloped single-stranded RNA β coronavirus.

With respect to the term “moderatelsevere cases of viral pneumonia”, based on the 5th edition of Guidelines for Diagnosis and Treatment of COVID-19 Infection in China of National Health Commission, the cases are categorized into 4 types:

(1) mild case, with slight clinical symptoms but imaging manifestations of pneumonia cannot be detected;

(2) moderate case, accompanied with fever, respiratory symptoms and imaging manifestations of pneumonia can be detected;

(3) severe case, referring to any one of the followings: respiratory distress having a respiration quotient. RR >30 times min, static oxygen saturation less than 93% or PaO₂/FiO₂ less than 300 mmHg (1 mmHg=0.133 kPa);

(4) critical case, having any one of the following situations: respiratory failure in need of mechanical ventilation and a breathing machine or other organ failures in need of ICU intensive care.

In this present disclosure, the moderate viral pneumonia includes the above “(2) moderate case”; the severe viral pneumonia includes the above “(3) severe case”and. “(4) critical case”.

The term “treatment”refers to: relief, prevention or reversal of a disease or symptom or at least one of differentiable symptom; improvement, prevention or reversal of at least one measurable body parameter associated with the treated disease or symptom; inhibition or mitigation in the progress of a disease or symptom, or delay in the attack of a disease or symptom. Treatment refers to clinical intervention implemented in clinical pathological process, which is expected to change the natural course of a disease for a subject and achieve prevention and treatment. Desirable therapeutic effects include but not limited. to prevention in occurrence or relapse of a disease, relief of symptom, weakening any direct or indirect pathological consequence of a disease, prevention in metastasis, reducing progression rate of a disease, improving or relieving the disease state as well as alleviating or improving prognosis. In some cases, medicaments (for example, AR inhibitor, and HIF-1α inhibitor) can be used to delay disease development or slow down progression. of a disease.

The term “prevention”refers to prophylaxis for the relapse or attack of one or more symptoms of a subject's disease generated by the administration of a preventive or a therapeutic agent. Healthy people are preventively administered a medicament (for example, AR inhibitor, and HIF-1α inhibitor in the present disclosure) to prevent the outbreak of the disease and symptom (for example, inflammatory cytokine storm or viral pneumonia, in the present disclosure. Moreover, the term “prevention”refers to preventively administering the medicament (AR inhibitor and/or HIF-1α inhibitor) of the present disclosure to a patient to be treated in the early stage. The “prevention”need not 100% eliminate the possibility of an incident. More accurately speaking, “prevention”refers to the decrease of the possibility of an incident in the presence of the compound or method.

The term “subject”or “individual”means a mammal subject. Exemplary subject includes but not limited to human, monkey, dog, cat, mouse, rat, cow, horse, camel, goat, rabbit and sheep. In some embodiments, the subject is human. In some embodiments, the subject suffers from a disease or disorder capable of being treated by the AR inhibitor and/or HIF-1α inhibitor provided herein. In some aspects, the disease or disorder is viral pneumonia, in some aspects, the disease or disorder is moderate or severe viral pneumonia.

The term “effective amount”refers to a sufficient drug dose capable of providing expected biological results. The results may be relief and/or remission of signs, symptoms or causes of a disease, or any other expected change in biological system. For example, the “effective amount”used for treatment is to provide an amount of the compound of this present disclosure required to significantly relieve a clinical disease. In any individual case, a proper “effective”amount may be determined by a person skilled in the art through conventional experiments. Therefore, the “effective amount”generally refers to the amount of active substances having therapeutical effects.

As used herein, the “medicament”refers to using a drug containing the HIF-1α inhibitor and AR inhibitor or a pharmaceutically acceptable pharmaceutical composition. Based on the medicament for treating viral pneumonia provided herein, the pharmaceutical composition contains the HIF-160 inhibitor and AR inhibitor and a pharmaceutically acceptable carrier or excipient. The medicament may be prepared into oral and non-oral preparations. Oral administration may be prepared into tablets, powders, granules, capsules and other conventional dosage forms; the excipient used may be one or more of starch, lactose, sucrose, mannose, and hydroxymethyl cellulose. A disintegrant may be one or more of potato starch, hydroxymethyl cellulose and the like. Adhesive may be one or more of Arabic gum, corn starch, gelatin, dextrin and the like. In addition to the above dosage forms, the oral preparation may be further made into emulsions, syrups, and the like. Non-oral preparation may be made into injections. Non-oral preparation may be made into injections with water for injection, normal saline, and glucose water, and may be also added with certain ratio of ethanol, propyl alcohol, ethylene glycol and the like.

The term “combined” used herein refers to using more than one preventives anchor therapeutic agents. The use of the “term” “combined”is free of limiting the administration sequence of the preventive and/or therapeutic agent to a subject suffering from a disease. The first preventive and/or therapeutic agent may be administered prior to (for example, prior to 1 min, 5 min, 15 min, 30 mM, 45 min, 1 h, 2 h, 4 h, 6 h, 12 h, 24 h, 48 h, 72 h, 96 h, 1 week, 2 weeks, 3 weeks, 4 weeks, 5 weeks, 6 weeks, 8 weeks or 12 weeks) the second preventive and/or therapeutic agent; simultaneously administered with the second preventive and/or therapeutic agent; or administered after (for example, 1 min, 5 min, 15 min, 30 min, 45 min, 1 h, 2 h, 4 h, 6 h, 12 h, 24 h, 48 h, 72 h, 96 h, 1 week, 2 weeks, 3 weeks, 4 weeks, 5 weeks, 6 weeks, 8 weeks or 12 weeks) the second preventive and/or therapeutic agent to a subject who has suffered from, currently suffers from or is susceptible to the disease). The preventive and/or therapeutic agent is administered to a subject such that the inhibitor(s) of the present disclosure may act with other agents alternately in sequence and time, thus providing enhanced effects relative to other administration modes. Any extra preventive and/or therapeutic agent may be administered with other extra preventives and/or therapeutic agents in any sequence.

The term “antibiotic”refers to molecules capable of inhibiting or killing microorganisms. The microorganisms include virus, bacteria, fungi or protozoa. Antibiotics include antiviral agents, antibacterial agents, antifungal agents and anfiprotozoan. Examples of antibiotic may include: (i) aminoglycosides, for example, gentamicin, amikacin, kanamycin, streptomycin, netilmicin, tobramycin, neomycin, paromycin (ii) ansamycins, for example, herbimycin. geldanamycin; (iii) carbacephems, for example, loracarbef; (iv) carbapenems, for example, ertapenum, imipenem/cilastatin, doripenem, meropenem; (v) the first-generation cephaolsporins, for example, cefadroxil, cefalexin, cefazolin, celalexin; (vi) the second-generation ceph.alosporins, for example, cefamandole, ceflaclor cefprozil, cefoxitin, cefuroxime; (vi) the third-generation cephalosporins, for example, cefixime cefditoren, cefoperazone, cefotaxime cefdinir, cefpodoxime, ceftibuten, ceftazidime, ceftizoxime, ceftriaxone; (vii) the fourth-generation cephalosporins, for example, cefepime; (viii) the fifth-generation cephalosporins, for example, ceftobiprole; (ix) glycopeptides, for example, teicoplanin, vancomycin; (x) macrolides, for example, axithromycin, clarithromycin, dirithromycine, erythromycin, troleandomycin, telithromyein, roxithromycin, spectinomycin; (xi) monobactams, axtreonam; (xii) penicilins, for example, amoxicillin, axlocillin, carbenicillin, ampicillin, cloxacillin, dicloxacillin, flucloxacillin, mezlocillin, nafcilin, oxacillin, meticitlin, penicillin, peperaeillin, ticarcillin; (xiii) antibiotic polypeptides, for example, bacitracin, colistin, polymyxin B; (xiv) quinolones, for example, ciprofloxacin, gatifloxacin, enoxacin, levofloxacin, lemefloxacin, moxifloxacin, norfloxacin, orfloxacin, trovafloxacin; (xv) sulfonamides, for example, mafenide, sulfacetamide, sulfamethizole, sulfanilamide, prontosil, sulfasalazine, sulfisoxazole, trimethoprim, trimethoprim-sulfamethoxazole (TMP-SMX)); (xvi) tetracyclines, for example, demeclocycline, minocycline, oxytetracycline doxycycline, tetracycline, and (xvii) others, for example, arspenarnine, chloramphenicol, clindamycin, ethambutol, lincomycin, fosfomycin, furazolidone, fusidic acid, isoniazid, metronidazole, mupirocin, nitrofurantoin, linezolid, platensimycin, pyrazinamide, quinupristin/dalfopristin, rifampin/rifampicin or tinidazole.

The term “medicator”usually may include: (i) an infusion module, the infusion module is used for administering a subject a pharmaceutical composition having active ingredients; (ii) a medicament used for infusion; the medicament contains active ingredients, and the active ingredients are selected from the following group: HIF-1α inhibitor, AR inhibitor, and a second therapeutic agent capable of being used for the treatment of viral pneumonia; and (iii) an optional efficacy monitoring module.

The present disclosure will be understood better according to the following examples. However, it is easy for a person skilled in the art to understand what are described in the examples are merely used to illustrate and explain the present disclosure, and are not to be construed as limiting the present disclosure as described in detail in the claims. Unless otherwise specified, reagents, methods and equipment used in the present disclosure are conventional methods; experimental materials used herein can be obtained from business companies.

### Example 1

Bioinfonnatics Seurat 3.0 and Single R software were used to analyze the transcriptome sequencing data of monoplasts in a severe COVID-19 patient and a subject. not infected with COVID-19 to find that the expression level of HIF-1α in lung fibroblasts of the infected was much higher than that in lung fibroblasts of the uninfected.

The transcriptome sequencing data of monoplasts in a. COVID-19 patient; GSM4516279, GSM4516280, GSM4516281, GSM4516282 data website: https://www.ncbi.nlm.nih.gov/geo.

The transcriptome sequencing data of monoplasts in the subject not infected with COVID-19: E-MTAB-6149 and E-MTAB-6653 data website: https://www.ebi.ac.uk/arrayexpress.

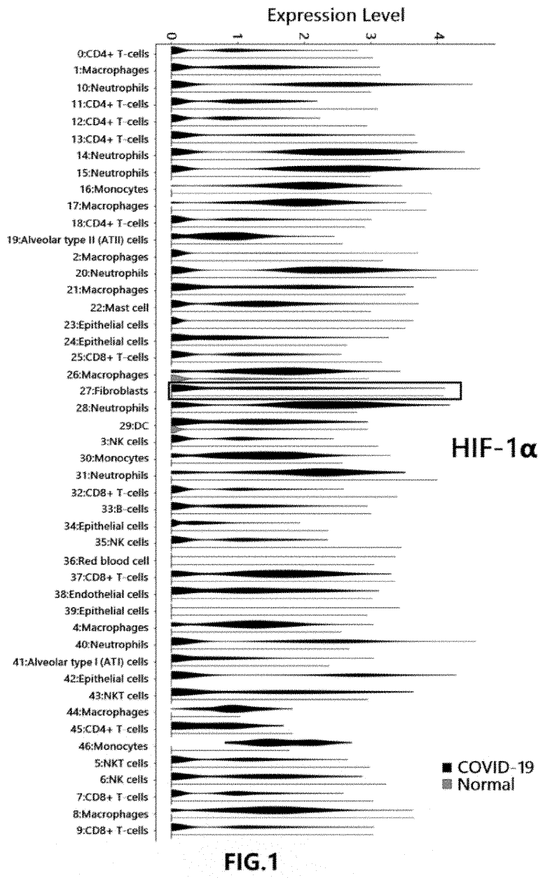

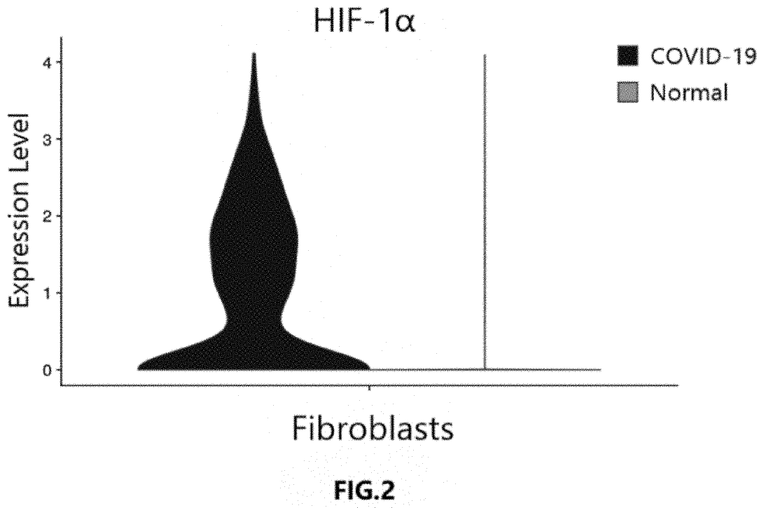

The analysis result is shown in FIG. 1, indicating the high expression of HIF-1α in lung fibroblasts of COVID-19 patients.

### Example 2

Bioinformatic analysis website tools TFmapper and UCSC Genome Browser were used to analyze the interaction situations of HIF-1α and AR in promoter regions of related genes (IL6, MMP2, MMP13, ADAMTS4, ELN, VCAN, COL3A1 and VEGFA). Bioinformatic analysis results indicate that HIF-1α and AR may bind to promoter regions of related genes (IL6, MMP2, MMP13, ADAMTS4, ELN, VCAN, COL3A1 and VEGFA), thus influencing the transcription of related genes.

### Example 3

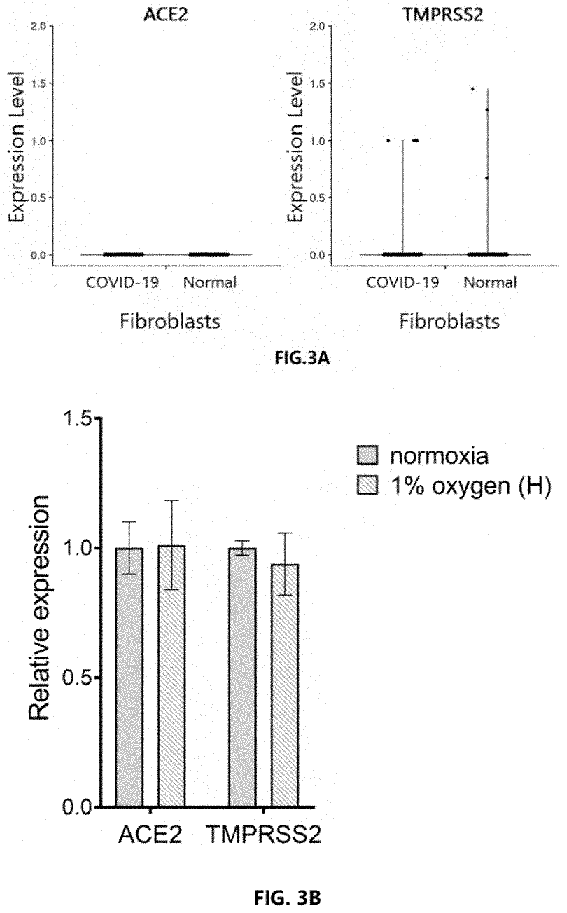

FIG. 3A shows expression levels of genes ACE2 and TMPRSS2 analyzed by analyzing the transcriptome sequencing data of monoplasts in a COVID-19 severe patient and a subject not infected with COVID-19 via bioinformatics software Seurat 3.0 and Single R.

The transcriptome sequencing data of monoplasts in a COVID-19 patient: GSM4516279, GSM4516280, GSM4516281, GSM4516282 data website: https://www.ncbi.nlm.nih.gov/geo.

The transcriptome sequencing data of monoplasts in the subject not infected with COV1D-19: E-MTAB-6149 and E-MTAB-6653 data website: https://www.ebi.ac.uk/arrayexpress.

The analysis results are shown in FIG. 3A. The experimental results indicate that the expression levels of genes ACE2 and TMPRSS2 are low in lung fibroblasts of the COVID-19 severe patient and a subject not infected with COVID-19, and there is no significant difference between the groups.

FIG. 3B shows that the gene expression levels of an angiotensin converting enzyme 2 (ACE2) and a transmembrane serine protease 2 (TMPRSS2) are explored by adding androgen (dihydrotestosterone) under activated AR pathway conditions. Under anoxic (37° C., 1% O₂) and normoxia conditions, human lung fibroblasts HFL1 were cultured in a culture medium added with dihydrotestosterone (10 nM, purchased from Sigma-Aldrich) for 48 h to collect cells, finally, real-time fluorescence quantification (qPCR) was perfbrmed to detect the expression levels the related genes.

The experimental results are shown in FIG. 3B and indicate that there are no significant differences of ACE2 and TMPRSS2 under anoxic and normoxia conditions in the lung fibroblasts under activated AR pathway conditions of cells.

To sum up, the transcriptome sequencing data of monoplasts in the severe COVID-19 patient and the subject not infected with COVID-19 are analyzed to find that the overall expression levels of the genes ACE2 and TMPRSS2 in fibroblasts are low and there are no significant differences between the injected and the uninfected. In vitro experiment researches also verify that there are no significant differences between the genomes ACE2 and TMPRSS2 in lung fibroblasts under low oxygen and normoxia conditions, which also exactly verifies that the genes ACE2 and TMPRSS2 do not participate in the inflammatory cytokine storm of a severe case.

### Example 4

Human lung fibroblasts HFL1 (purchased from Procell, Art. No.: CL-0106) were cultured on an HFL1 culture medium (Ham's F-12K+10% FBS+1% P/S) under low oxygen conditions (culture conditions of a dedicated low-oxygen incubator: 37° C., 1% O₂), and then subjected to protein extraction with a RIPA tissue/cell lysis solution and a PMSF reagent; then a Pierce™ Co-Immunoprecipitation Kit was used for a co-immunoprecipitation (co-IP) experiment according to the selectivity action principle between antibodies and antigens; afterwards, the result obtained from the co-immtmoprecipitation was further subjected to the next Western bloting experiment.

Specific steps of the Western bloting experiment were as follows: firstly, a 10% SDS separation gel and spacer gel were prepared according to the formula; samples were mixed with a loading buffer solution, and boiled for 5 min in a 100° C. ice bath and centrifuged, same amount of the obtained solution was added to each lane with a micropipette for electrophoretic separation, and subjected to protein isolation by SDS-PAGE, and then transferred to a PVDP membrane (Merck Millipore, Mass., USA) and incubated for 1 h in 5% BSA. The membrane was incubated over the night with primary antibodies HIF-1α and AR diluted at 1:400 at 4° C., and washed for 3 times with TBST and incubated for 1 h with a goat anti-rabbit second antibody. Then the obtained membrane was washed for 3 times with a PBS buffer solution at room temperature, 5 min for each time. The membrane was immersed into an ECL reaction solution for 1 min at room temperature. After removing liquid, the membrane was covered by a food wrapper, and subjected to ray exposure, developing and photographic fixing in a dark room to observe the result.

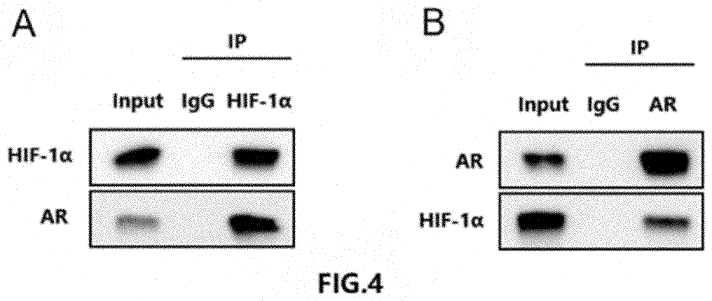

The results are shown in FIGS. 4A-4B. The experimental results show that in human lung fibroblasts, HIF-1α interacts with AR.

### Example 5

Lung fibroblasts HFL1 (purchased from Procell, Art.No.: CL-0106) were cultured under normoxia 37° C., 5% CO₂) and low oxygen (37° C., 1% O₂) conditions, and then treated by an HIF-1α inhibitor or an AR inhibitor respectively or a combination thereof 48 h later, cells were collected and treated, thus detecting the expression levels of the related genes by real-time fluorescence quantification PCR (qPCR).

The HIF-1α inhibitor KC7F2, LW 6, PX-478 2HCl (purchased from Selleck. Art.No.: S7946, S8441, S7612). The AR inhibitor Enzalutamide, Apalutamide, Darolutamide, Galeterone, AZD3514 (purchased from Selleck, Art.No.: S1250, S2840, S7559, S2803, S7040). Proxalutamide (from a patent invention, publication number: CN106810542A), SHR-3680 (from a patent invention, publication number: CN103958480B). The dedicated HFL1 cell medium (purchased from Procell, Art.No.: CM-0106). The inhibitor ingredients added to different treatment groups are shown in Table 1.

TABLE 1
Inhibitor ingredient added to groups 1-14

| Group | Inhibitor ingredient |
|---|---|
| Group 1 | No HIF-1α inhibitor or AR inhibitor |
| Group 2 | HIF-1α inhibitor 1, KC7F2 20 μM. (Purchased from |
| | Selleck, Art. No.: S7946) |
| Group 3 | HIF-1α inhibitor 2, LW 6 20 μM. (purchased from |
| | Selleck, Art. No.: S8441) |
| Group 4 | HIF-1α inhibitor 3, PX-478 2HCI, 20 μM. (Purchased from |
| | Selleck, Art. No.; S7612) |
| Group 5 | AR inhibitor 1, Enzalutamide 10 μM. (Purchased from |
| | Selleck, Art. No.: S1250) |
| Group 6 | AR inhibitor 2, Apalutamide 10 μM. (Purchased from |
| | Selleck, Art. No.: S2840) |
| Group 7 | AR inhibitor 3, Darolutamide 10 μM. (Purchased from |
| | Selleck, Art. No.: S7559) |
| Group 8 | AR inhibitor 4, Galeterone 10 μM. (Purchased from |
| | Selleck, Art. No.: S2803) |
| Group 9 | AR inhibitor 5, AZD3514 10 μM. (Purchased from |
| | Selleck, Art. No.: S7040) |
| Group 10 | AR inhibitor 6, Proxalutamide 10 μM. (From a patent |
| | invention, publication number: CN106810542A) |
| Group 11 | AR inhibitor 7, SHR-3680 10 μM. (From a patent |
| | invention, publication number: CN103958480B) |
| Group 12 | Combined HIF-1α inhibitor 1 (20 μM) and AR inhibitor 1 |
| | (10 μM) treatment 1 |
| Group 13 | Combined HIF-1α inhibitor 2 (20 μM) and AR inhibitor 6 |
| | (10 μM) treatment 2 |
| Group 14 | Combined HIF-1α inhibitor 3 (20 μM) and AR inhibitor 7 |
| | (10 μM) treatment 3 |

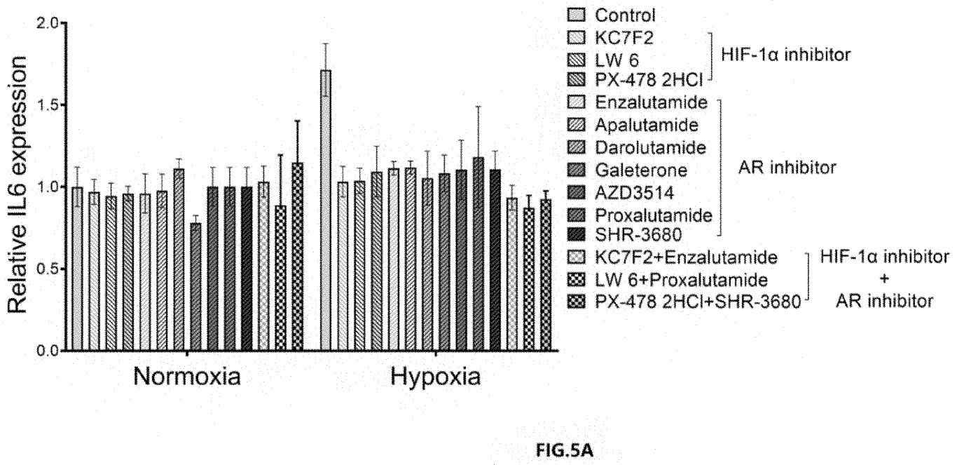

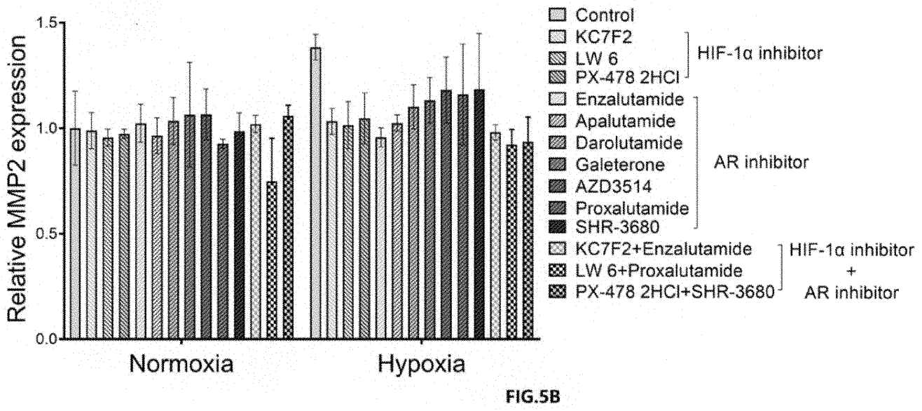

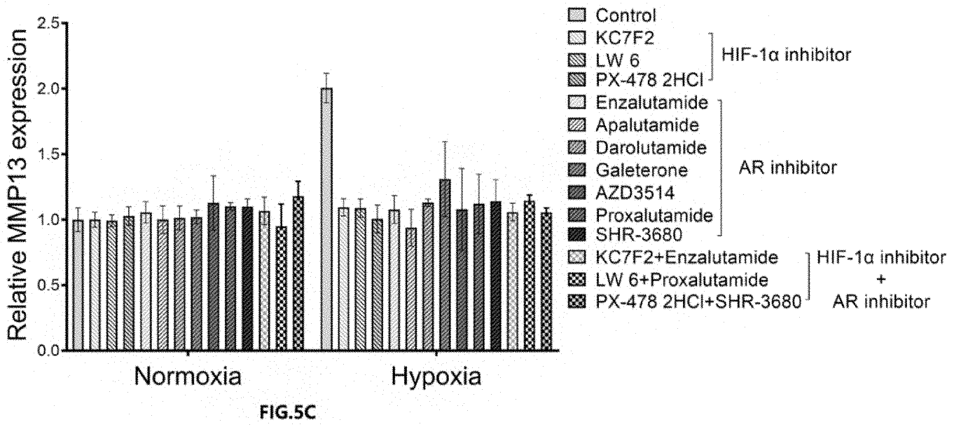

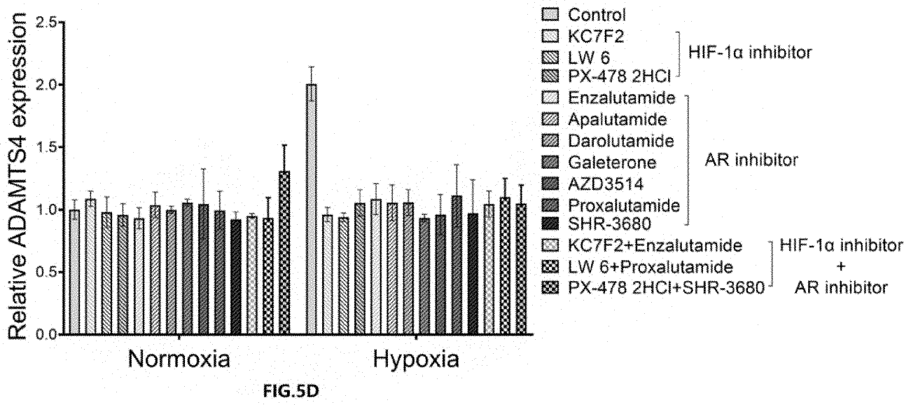

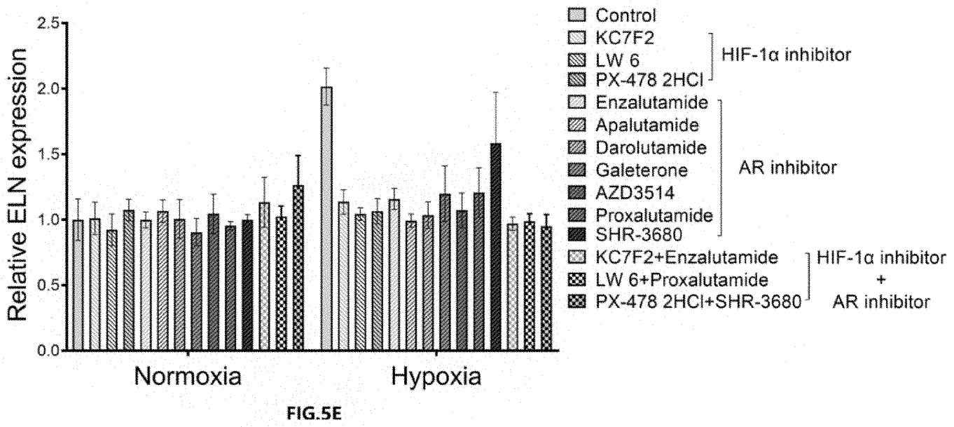

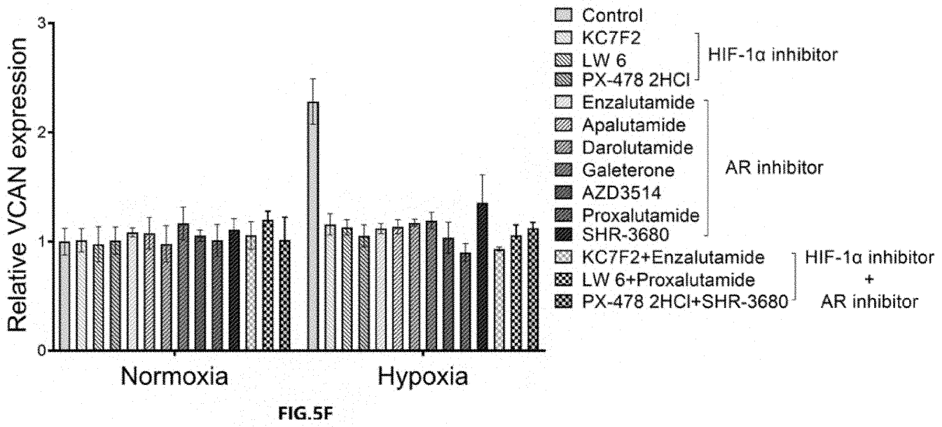

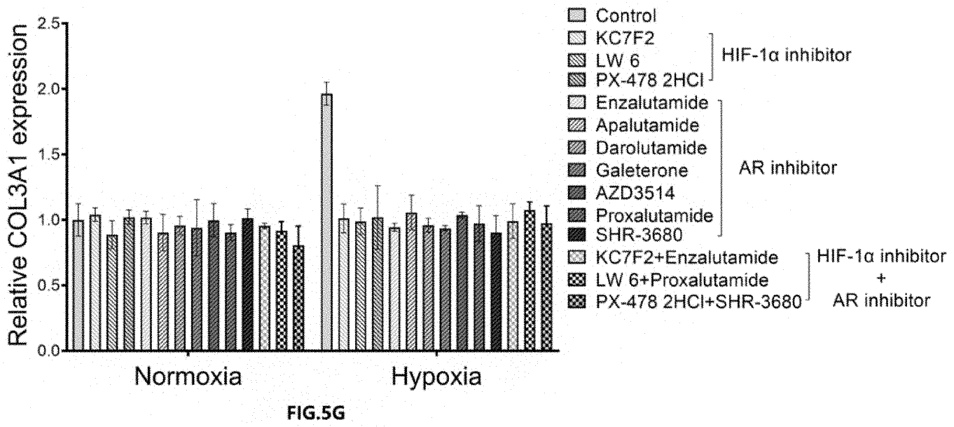

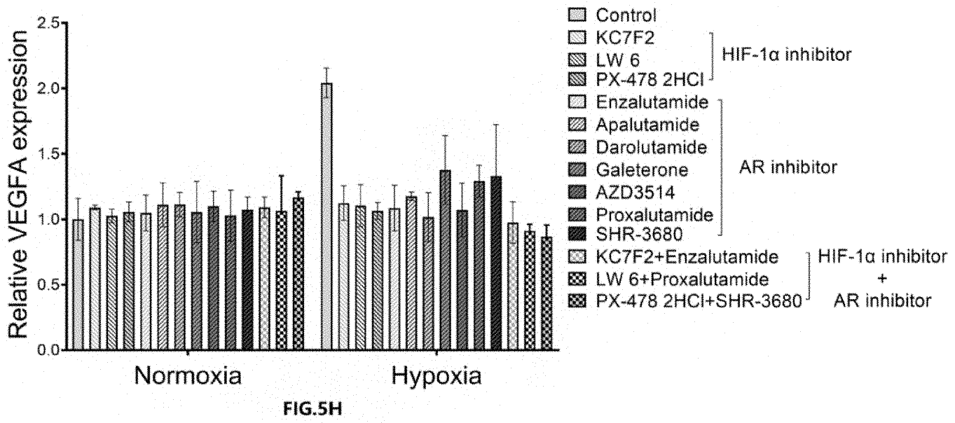

The experimental results are shoi Fn in FIGS. 5A-5H. he two inhibitors do not influence the expression levels of the related genes (IL6, ELN, VCAN, VEGFA, MMP2, ADAMTS4, MMP13 and COL3AS1) under normoxia conditions. However, relative to the normoxia conditions, the expression levels of the related genes (IL6, ELN, VCAN, VEGFA, MMP2, ADAMTS4, MMP13 and COL3AS1) increase significantly under low oxygen conditions. Moreover, the two inhibitors may Obviously decrease the expression levels of the related genes (IL6, ELN, VCAN, VEGFA, MMP2, ADAMTS4, MMP13 and COL3AS1) under low oxygen conditions. The result specifically indicates that the single treatment group of the HIF-1α inhibitor, the single treatment group of the AR inhibitor, as well as the combined treatment group of the HIF-1α inhibitor and the AR inhibitor may effectively inhibit the expression of the related genes. The result indicates that the inhibitors HIF-1α and AR may inhibit the expression of HIF-1α and AR, thus influencing the interaction between the two on the promoter regions of the related genes, thereby influencing the transcription process of the related genes and reducing the expression levels of the related genes.

### Example 6

Androgen (dihydrotestosterone) was added to explore the regulationof the inhibitor on related genes under activated AR pathway conditions.

Under anoxic (37° C., 1% O₂) conditions, human lung fibroblasts HFL1 which were treated by different groups of inhibitors (for example, as shown in Table 1 of Example 5) were cultured in a culture medium added with dihydrotestosterone (10 nM, purchased from Sigma-Aldrich) for 48 h to collect cells, finally, real-time fluorescence quantification (qPCR) was performed to detect the expression levels the related genes.

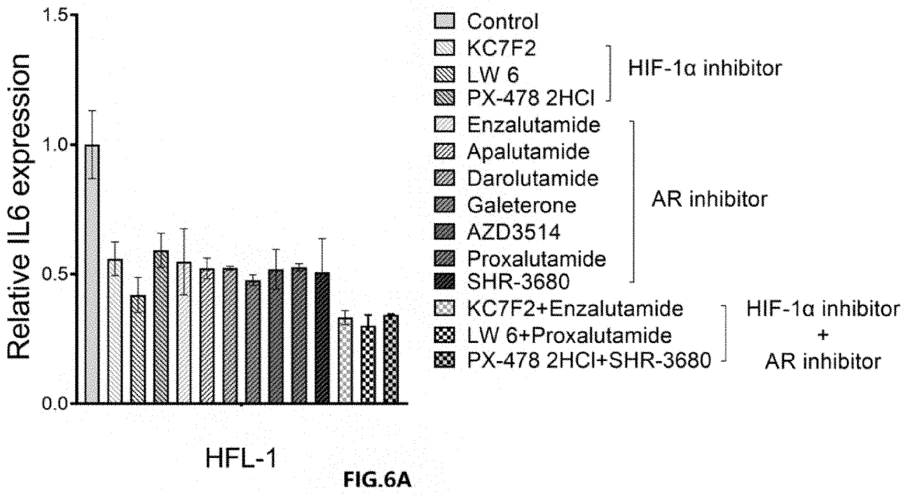

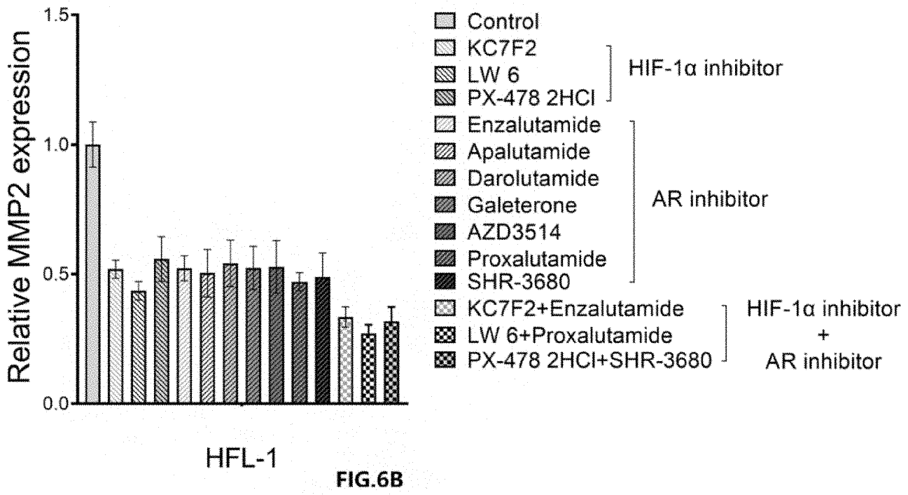

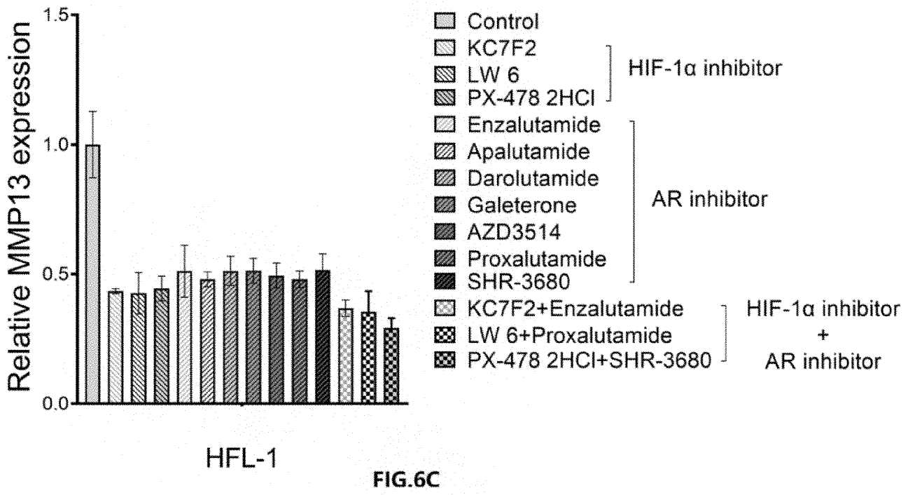

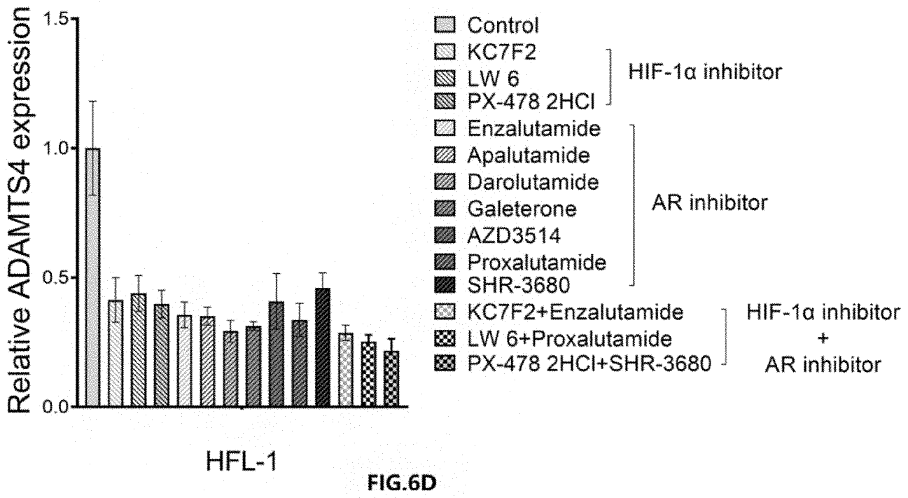

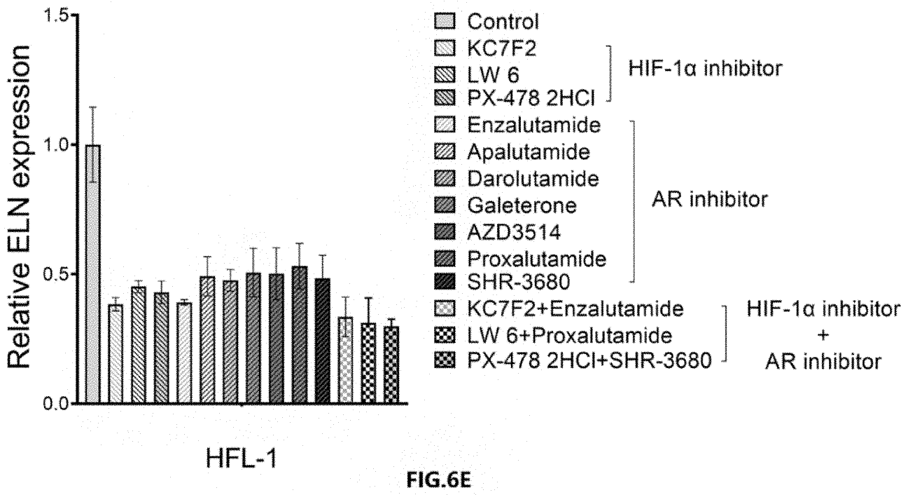

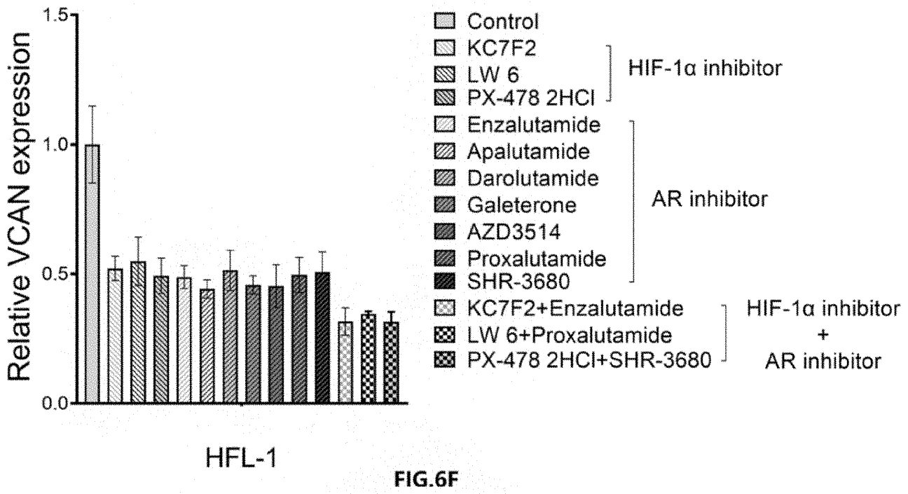

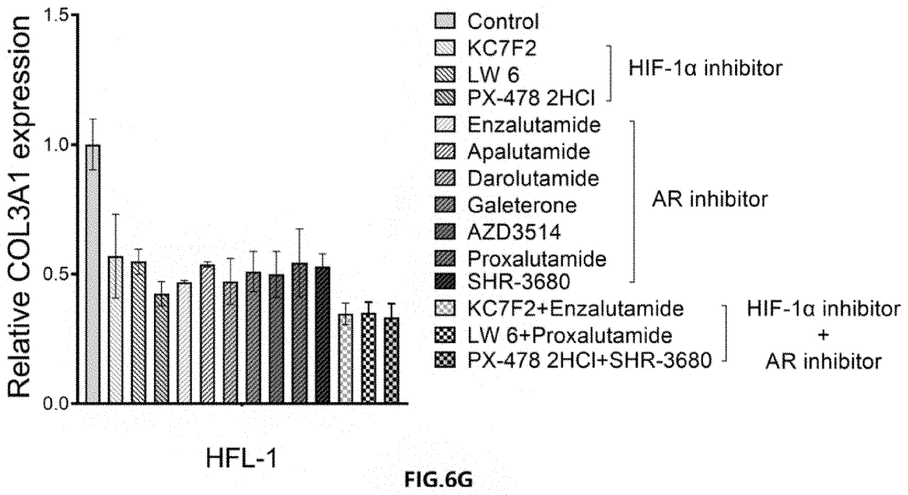

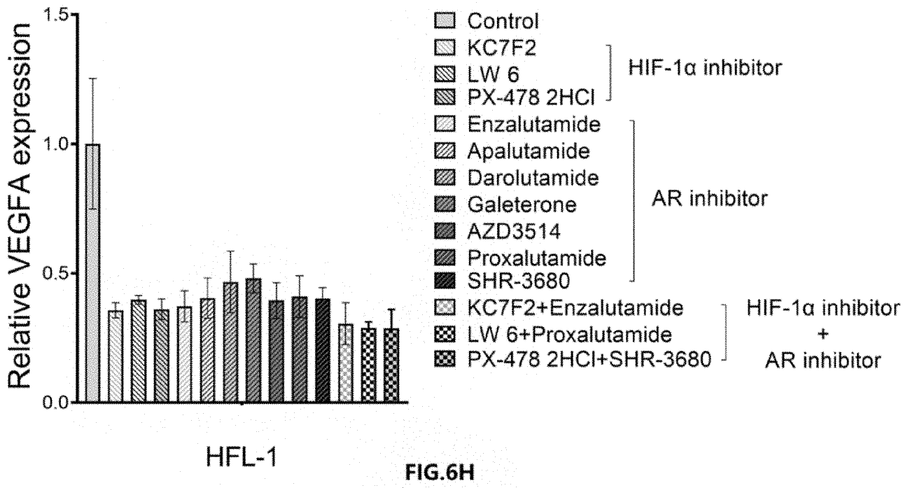

The experimental results are shown in FIGS. 6A-6H. The two inhibitors may effectively reduce the expression levels of the related genes (IL6, MMP2, MMP13, ADAMTS4, ELN, VCAN, COL3A1 and VEGFA) under low oxygen and activated AR signal pathway conditions. The results indicate that when HIF-1α inhibitor may influence the expression of the related genes by inhibiting self-gene expression in lung fibroblasts under the activated AR signal pathway conditions; meanwhile, the AR inhibitor may influence the AR signal pathway; thus effectively inhibiting the expression of the related genes.

### Example 7

A viral pneumonia mouse model was established to explore the influences of the HIFI a inhibitor and AR inhibitor on the mouse viral pneumonia.

Establishment of the viral pneumonia mouse model: male C57BL/6 mice with 6-8 weeks old and body weight of 18-22 g were selected for model preparation, Experimental animals were randomly divided into the following groups:

| Group | Treatment mode |
|---|---|
| Group 1 | Normal group |
| Group 2 | Virus group, no HIF-1α inhibitor or AR inhibitor |
| Group 3 | Mice were intragastrically administered with the HIF-1α |
| | inhibitor 1, LW 6 10 mg/kg once per day for 5 consecutive days. |
| Group 4 | Mice were intraperitoneally administered with the HIF-1α |
| | inhibitor 2, PX-478 2HCI, 100 mg/kg for once. |
| Group 5 | Mice were intraperitoneally administered with the AR |
| | inhibitor 1, Proxalutamide, 100 mg/kg for once. |
| Group 6 | Mice were intragastrically administered with the AR |
| | inhibitor 2. SHR-3680, 5 mg/kg once per day for 5 |
| | consecutive days. |
| Group 7 | LW 6 (mice were intragastrically administered 10 mg/kg, |
| | once per day for 5 consecutive days) in combination with |
| | SHR-3680 (mice were intragastrically administered 5 mg/kg |
| | once per day for 5 consecutive days) |

Mice in the normal control group were narcotized mildly with 5% chloral hydrate, and subjected to nasal inhalation of 50 μl PBS; the rest groups of mice were narcotized mildly with chloral hydrate, and instilled with 50 μl influenza A Hp1N1 virus 10 times the infective dose of LD50. Mice in the administration. team were administered according to the above solution 24 h later after modeling. The survival conditions of the mice were observed for consecutive 14 d after virus attack to make statistics on the death situation and body weight variation of the mice,

TABLE 2
Statistics on mean days of mice survival and survival rate

| | Mean days of | Survival | Survival |
|---|---|---|---|
| Group | survival | number (pcs.) | rate (%) |
| Normal group | 14 | 10 | 100 |
| Virus control | 9.1 ± 2.21 | 0 | 0 |
| LW6 | 11.5 ± 2.54 | 5 | 50 |
| PX-478 2HCI | 11.9 ± 2.81 | 6 | 60 |
| Proxalutamide | 11.0 ± 2.83 | 4 | 40 |
| SHR-3680 | 11.3 ± 2.87 | 5 | 50 |
| LW6 + SHR-3680 | 12.9 ± 2.21 | 8 | 80 |

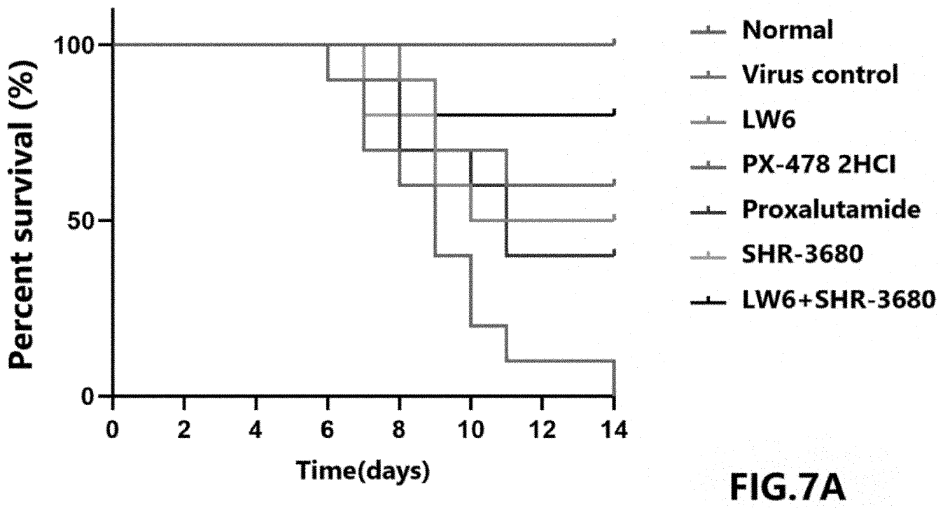

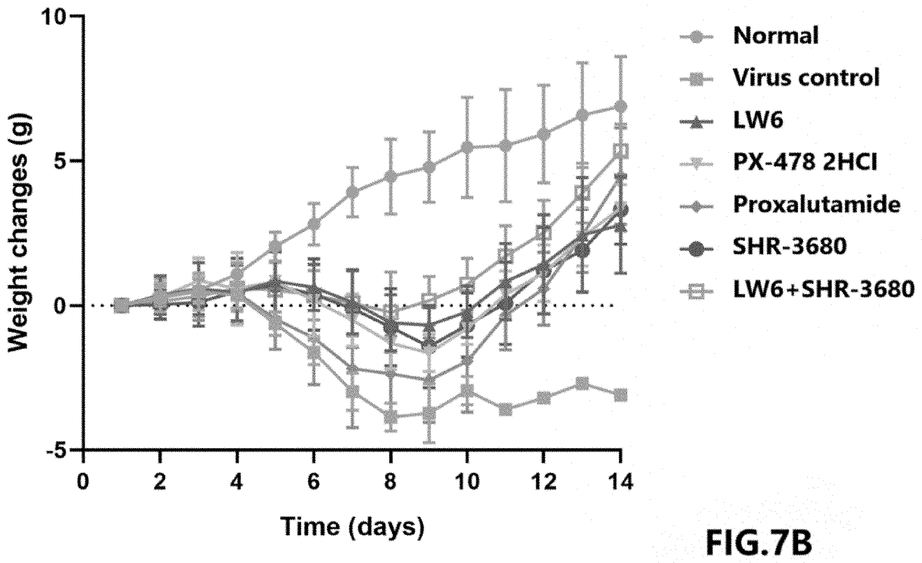

The experimental results shown in FIGS. 7A-B and Table 2 are as follows: 14 d survival rate of the mice in the normal control goup is 100%; 14 d survival rate of the mice in the virus control group is 10%; 14 d survival rate of the mice in the HIF-1α a inhibitor groups 1 and 2, and the AR inhibitor groups 1 and 2 are respectively 50%, 60%, 40% and 50%. Meanwhile, the administration of the HIF-1α inhibitor and/or AR inhibitor slows down the loss of mice weight to different extents, and extends the days of mice survival. The results indicate that the treatment groups of the HIF-1α inhibitor and/or AR inhibitor significantly improve the survival rate of model mice.

Although basic principles, major features and advantages of the present disclosure have been shown and described above, those skilled in the art shall appreciate that the present disclosure is not limited to the above examples; and what is described in the above examples and the desciption is merely for illustration of the principle of the present disclosure. Nevertheless, there may be various further changes and impovements of the present disclosure without departing from the spirit and scope thereof; and these changes and improvements shall fall within the protection scope of the present disclosure.

1. A medicament for inhibiting an inflammatory cytokine storm including an inhibitor, selected from the group consisting of an AR inhibitor and an HIF-1α inhibitor, wherein the AR inhibitor and/or the HIF-1α inhibitor are adapted to inhibit the following in fibroblasts:
gene expression activity of AR,
gene expression activity of HIF-1α, and
formation of an AR and HIF-1α complex.

2. The medicament of claim 1, wherein the inhibitor is adapted to inhibit fonnation of an AR and HIF-1α complex in fibroblasts to influence expression of an inflammation-related gene, wherein the inflammation-related gene is selected from the group consisting of IL6, MMP2, MM13, ADAMTS4, ELN. VCAN, COL3A1 and VEGFA.

3. The medicament of claim 1, wherein the inflammatory cytokine storm is caused by viral pneumonia.

4. The medicament of claim 3, wherein the viral pneumonia is moderate or severe.

5. The medicament of claim 1, wherein the HIF-1α inhibitor is selected from one or more of KC7F2, LW 6 and PX-478 2HCl.

6. The medicament of claim 1, wherein the AR inhibitor is selected from one or more of Enzalutamide, Apalutamide, Darolutamide, Proxalutamide, Galeterone, AZD3514 and SHR-3680.

7. A medicament for preventing or treating viral pneumonia including an inhibitor, selected from the group consisting of an AR inhibitor and an HIF-1α inhibitor

8. The medicament of claim 7, wherein the viral pneumonia is selected from the group consisting of respiratory syncytial virus pneumonia, influenza A virus pneumonia and COVID-19.

9. The medicament of claim 7, wherein the viral pneumonia is moderate or severe.

10. The medicament of claim 7, wherein the HIF-1α inhibitor is selected from the group consisting of KC7F2, LW 6 and PX-478 2HCl.

11. The medicament of claim 7, wherein the AR inhibitor is selected from the group consisting of Enzalutamide, Apalutamide, Darolutamide, Proxalutamide, Galeterone, AZD3514 and SHR-3680.

12. A method for treating or preventing viral pneumonia, comprising administering a subject in need an effective amount of an inhibitor selected from the group consisting of an AR inhibitor and an HIF-1α inhibitor.

13. The method of claim 12, wherein the viral pneumonia is selected from the group consisting of respiratory syncytial virus pneumonia, influenza A virus pneumonia and COVID-19.

14. The method of claim 12, wherein the viral pneumonia is moderate or severe.

15. The method of claim 12, wherein the HIF-1α inhibitor is selected from the group consisting of KC7F2, LW 6 and PX-478 2HCl; and the AR inhibitor is selected from one or more of Enzalutamide, Apalutamide, Darolutamide, Proxalutamide, Galeterone, AZD3514 and SHR-3680.

16. A medicator, comprising a component configured to hold an inhibitor selected from the group consisting of an AR inhibitor and a HIF-1α inhibitor for administration to a subject.

17. The medicator of claim 16, wherein the component is selected from the group consisting of a syringe, an infusion set and an implantable medicator.

18. The medicator of claim 16, further comprising:
an infusion module configured to administer the inhibitor; and
an efficacy monitoring module.

19. The method of claim 12, wherein the AR inhibitor and/or the HIF-1α inhibitor are adapted to inhibit the following in fibroblasts:
gene expression activity of AR,
gene expression activity of HIF-1α, and
formation of an AR and HIF-1α complex

20. The method of claim 12, wherein the inhibitor is adapted to inhibit formation of an AR and HIF-1α complex in fibroblasts to influence expression of an inflammation-related gene, wherein the inflammation-related gene is selected from the group consisting of IL6, MMP2, MMP13, ADAMTS4, ELN, VCAN, COL3A1 and VEGFA.
